(19) United States
(12) Patent Application Publication
(10) Pub. No.: US 2021/0015403 A1
(43) Pub. Date: Jan. 21, 2021

(54) ANESTHETIZING MONITORING SYSTEM, UNIT AND METHOD THEREFORE

(71) Applicant: SENZIME AB (PUBL.), UPPSALA (SE)

(72) Inventor: John Martyn GRAY, Leeds Yorkshire (GB)

(21) Appl. No.: 16/980,882

(22) PCT Filed: Mar. 11, 2019
(86) PCT No.: PCT/SE2019/050219

§ 371 Date: Sep. 15, 2020

(30) Foreign Application Priority Data
Mar. 16, 2018 (SE) 1850295-5

(51) Int. Cl. A61B 5/11; A61B 5/0492; A61B 5/00

(52) U.S. Cl. CPC A61B 5/1106; A61B 5/7275; A61B 5/0492

(57) ABSTRACT

The invention relates to a method (400) performed by an anesthetizing monitoring unit (110) configured to determine an anesthetized patient state, the method comprising disabling (410) an input port (111) of the anesthetizing monitoring unit (110), transmitting (420) a stimuli signal (Sₛₜᵢₘᵤₗᵢ) using an output port (112) of the anesthetizing monitoring unit (110), enabling (430) the input port (111) of the anesthetizing monitoring unit (110) with a delay (ΔT) relative to the transmission of the stimuli signal (Sₛₜᵢₘᵤₗᵢ), receiving (440) an evoked electromyography, EMG, response signal (S_{Response}) in response to the transmitted stimuli signal (Sₛₜᵢₘᵤₗᵢ), determine (450) an anesthetized patient state by determining a neuromuscular function value using properties of the stimuli signal (Sₛₜᵢₘᵤₗᵢ) and the response signal (S_{Response}). The invention further relates to an anesthetizing monitoring unit (110) and an anesthetizing monitoring system (100).

### TECHNICAL FIELD

The present disclosure relates to anesthetizing monitoring systems, in particular anesthetizing monitoring systems used to determine an anesthetized patient state.

### BACKGROUND

In hospitals around the world, patients are administered muscle relaxants (also called neuromuscular blocking agents, NMBAs, which inhibit neuromuscular transmission. These relaxant agents decrease muscle tension and suppress reflex contractions. In particular non-depolarizing agents of NMBAs have an effect only for a certain number of minutes, so they may have to be administered repeatedly throughout a surgical procedure.

Drug effects must completely dissipate once the surgical procedure is complete and the patient is in recovery, e.g. so that patients can start breathing on their own (spontaneously). Reversal drugs (e.g. anticholinesterases) can be administered to speed-up recovery from muscle relaxants, but must also be administered in a controlled manner over time as reversal drugs can slow the heart to dangerous levels (bradycardia), and can have a host of other unpleasant side effects.

Neuromuscular monitoring systems using evoked electromyography, EMG, have been proposed to give an indication of the degree of neuromuscular block. Evoked EMG involves sending a stimulus signal to stimulating electrodes positioned on the patient's body and monitoring the response from receiving electrodes also positioned on the patient's body.

An example of such a system is provided in the document EP0025222 A2, which relates to the technical field of determining a degree of neuromuscular blockage, and shows a device providing an indication of muscular blockage.

A problem with such systems is that the EMG response signal may comprise an artifact or non-linear offset of the amplitude, which may degrade the linearity of the measurement or if the artifact varies with time will degrade the accuracy of the determination of the muscular block. In one example, the artifact appears as an additive signal to the response signal such that the amplitude, e.g. voltage, power or amplitude, of the response signal appears with varying amplitude which is not caused by a change in the state of the patient.

Thus, there is a need for an improved system, unit and method for anesthetizing monitoring.

### OBJECTS OF THE INVENTION

An objective of embodiments of the present invention is to provide a solution which mitigates or solves the drawbacks described above.

### SUMMARY OF THE INVENTION

The above and further objectives are achieved by the subject matter described herein. Further advantageous implementation forms of the invention are described herein.

According to a first aspect of the invention, the above mentioned objectives are achieved by a method performed by an anesthetizing monitoring unit configured to determine an anesthetized patient state, the method comprising disabling an input port of the anesthetizing monitoring unit, transmitting a stimuli signal using an output port of the anesthetizing monitoring unit, enabling the input port of the anesthetizing monitoring unit with a delay relative to the transmission of the stimuli signal, receiving an evoked electromyography, EMG, response signal in response to the transmitted stimuli signal, determine an anesthetized patient state by determining a neuromuscular function value using properties of the stimuli signal and the response signal.

At least one advantage of this embodiment is that an improved anesthetized patient state can be determined by disabling and enabling the input port before and after a stimuli pulse to eliminate or reduce the impact of time variant amplitude artifacts.

According to a second aspect of the invention, the above mentioned objectives are achieved by an anesthetizing monitoring unit configured to determine an anesthetized patient state, the anesthetizing monitoring unit comprising an input port, an output port, and processing circuitry being configured to perform the method according to the first aspect.

According to a third aspect of the invention, the above mentioned objectives are achieved by an anesthetizing monitoring system configured to determine an anesthetized patient state, the anesthetizing monitoring system comprising an anesthetizing monitoring unit comprising an input port and an output port, stimulating electrodes electrically coupled to the output port and being configured to receive a stimuli signal from the output port and deliver the stimuli signal to the anesthetized patient, receiving electrodes electrically coupled to the input port and being configured to obtain an evoked electromyography, EMG, response signal, in response to the stimuli signal, from the anesthetized patient, the anesthetizing monitoring unit being configured to perform the method according to the first aspect.

The advantages of the second and third aspects are at least the same as for the first aspect.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly

### BRIEF DESCRIPTION OF THE DRAWINGS

FIG. 1 shows an anesthetizing monitoring system according to one or more embodiments of the present invention.

FIG. 2A illustrates a signal diagram of a stimuli signal according to one or more embodiments of the present invention.

FIG. 2B illustrates a signal diagram of a response signal according to one or more embodiments of the present invention.

FIG. 3 illustrates an anesthetizing monitoring unit according to one or more embodiments of the present invention.

FIG. 4 shows a block diagram of a method according to one or more embodiments of the present invention.

FIG. 5 illustrates an anesthetizing monitoring unit according to one or more embodiments of the present invention.

A more complete understanding of embodiments of the invention will be afforded to those skilled in the art, as well as a realization of additional advantages thereof, by a consideration of the following detailed description of one or more embodiments. It should be appreciated that like reference numerals are used to identify like elements illustrated in one or more of the figures.

### DETAILED DESCRIPTION

An “or” in this description and the corresponding claims is to be understood as a mathematical OR which covers “and” and “or”, and is not to be understand as an XOR (exclusive OR). The indefinite article “a” in this disclosure and claims is not limited to “one” and can also be understood as “one or more”, i.e., plural.

The term anesthetizing monitoring unit signifies herein a unit comprising processing circuitry, such as a processor and coupled memory, adapted for or suitable to be used in a hospital environment, e.g. when performing or recovering from surgery. Examples may include a dedicated computer system, an Electronic Control Unit, a server, a tablet, a smart watch or a smartphone.

The term stimuli signal signifies herein a signal delivered to an anesthetized patient in order to stimulate a motor nerve. The stimuli signal may e.g. be in the form of a pulse in a pulse wave or pulse train pulse or a plurality of pulse wave or pulse train pulses having amplitude A_{Stimuli}. The stimuli signal S_{Stimuli} may be delivered a constant current. The stimuli signal is typically delivered to stimulating electrodes 122 attached to an anesthetized patient 130.

The term evoked electromyography, EMG, response signal S_{Response} signifies herein a signal received in response to the transmitted stimuli signal S_{Stimuli}. The response signal may e.g. be in the form of a sinusoidal signal, a pulse wave or a pulse train pulse or a plurality of pulse wave or pulse train pulses having amplitude A_{Response}. The response signal is typically obtained from receiving electrodes 121 attached to an anesthetized patient 130.

In one example, a subject having been administered a muscle relaxant agent includes stimulating a motor nerve with stimuli signal. After each stimulus of the motor nerve, the muscle response in the muscle(s) innervated by the stimulated motor nerve is recorded as a response signal S_{Response}, e.g. to provide an assessment of neuromuscular function or blockade in the subject. Each stimuli signal is sufficient to cause an evoked muscle response signal under normal physiological conditions. As muscle relaxants are administered to a subject, the amplitude of the evoked muscle response signal decreases relative historical or previously detected response signals or no response signal is detected.

The term “configured to” may be used interchangeably with “adapted to” or “operative to” in the disclosure herein.

The term “memory” may be used interchangeably with “computer readable medium” or “non-transitory computer readable medium” in the disclosure herein.

Provided in the present disclosure are systems, units and methods for determine an anesthetized patient state by determining a neuromuscular function value, e.g. by monitoring neuromuscular blockade of muscles in patients being administered muscle relaxants such as a neuromuscular blocking agent and/or a depolarizing agent and/or a non-depolarizing agent.

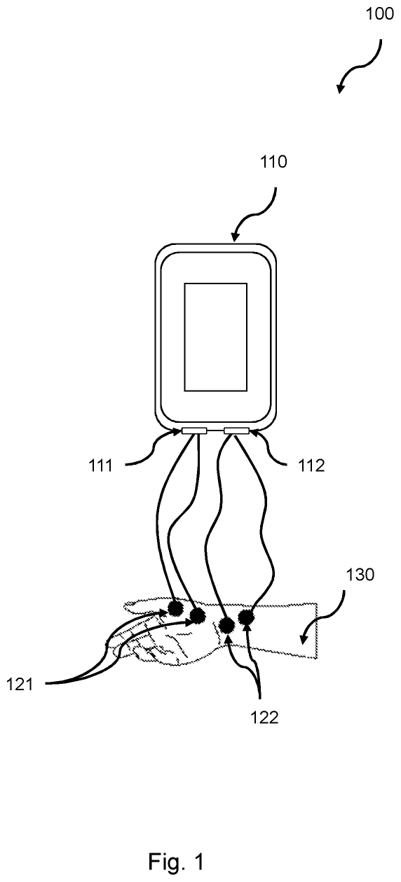

FIG. 1 shows an anesthetizing monitoring system 100 according to one or more embodiments of the present invention. The anesthetizing monitoring system 100 may comprise an anesthetizing monitoring unit 110 provided with an input port 111 and an output port 112 according to embodiments described herein. The anesthetizing monitoring system 100 may further comprise stimulating electrodes 122 configured to be electrically coupled to the output port 112 and being configured to receive a stimuli signal S_{Stimuli}, from the output port 112 and deliver the stimuli signal S_{Stimuli}, to the anesthetized patient 130. The anesthetizing monitoring system 100 may further comprise receiving electrodes 121 configured to be electrically coupled to the input port 111 and being configured to obtain an evoked electromyography, EMG, response signal S_{Response}, in response to the stimuli signal S_{Stimuli}, from the anesthetized patient 130.

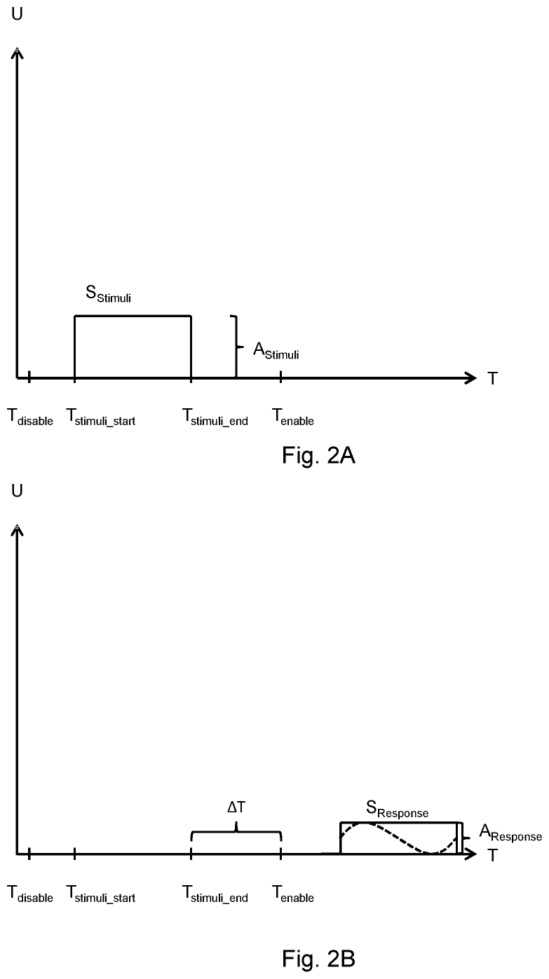

FIG. 2A illustrates a signal diagram of a stimuli signal according to one or more embodiments of the present invention. Voltage amplitude (U) is shown on the vertical axis of the diagram and time (T) is shown on the horizontal axis of the diagram. At a first time instant T_{disable}, the input port 111 of the anesthetizing monitoring unit 110 is disabled. The stimuli signal S_{Stimluli}, is then transmitted using the output port 112 of the anesthetizing monitoring unit 110. The stimuli signal S_{Stimuli} has amplitude of A_{Stimuli}, e.g. voltage amplitude. As described further in relation to FIG. 1, the stimuli signal S_{Stimuli}, is delivered to the anesthetized patient 130 by stimulating electrodes 122 attached to the anesthetized patient 130. The stimuli signal S_{Stimuli}, may comprise a single pulse, such as a pulse of a pulse wave or pulse train, or a plurality of pulses also referred to as a repeated pulse train.

FIG. 2B illustrates a signal diagram of a response signal according to one or more embodiments of the present invention. Voltage amplitude (U) is shown on the vertical axis of the diagram and time (T) is shown on the horizontal axis of the diagram. After transmission of the stimuli signal S_{Stimuli} and/or a pulse comprised in the stimuli signal S_{Stimuli}, the input port 111 of the anesthetizing monitoring unit 110 is enabled at time T_{enable} with a delay AT relative to the end of transmission of the stimuli signal S_{Stimuli} and/or a pulse comprised in the stimuli signal S_{Stimuli}. In a first example, the input port 111 of the anesthetizing monitoring unit 110 is enabled at time T_{enable} relative to the end time T_{stimuli_end} of the single pulse, i.e. at time T_{stimuli_end}+ΔT. In a further example, the input port 111 of the anesthetizing monitoring unit 110 is enabled relative to the end time T_{stimuli_end} one of the pulses of the repeated pulse train. An an evoked electromyography, EMG, response signal S_{Response} in response to the transmitted stimuli signal S_{Stimuli}, is then received. The response signal S_{Response} may be in the form of a pulse having amplitude A_{Response} or a single cycle of a sinusoid signal, with the amplitude A_{Response} being measured between the positive and negative peak of the sinusoid signal. The response signal S_{Response} has ideally either constant amplitude A_{Response} when having a pulse shape or follows the amplitude of a typical sinusoid signal when having the sinusoidal shape.

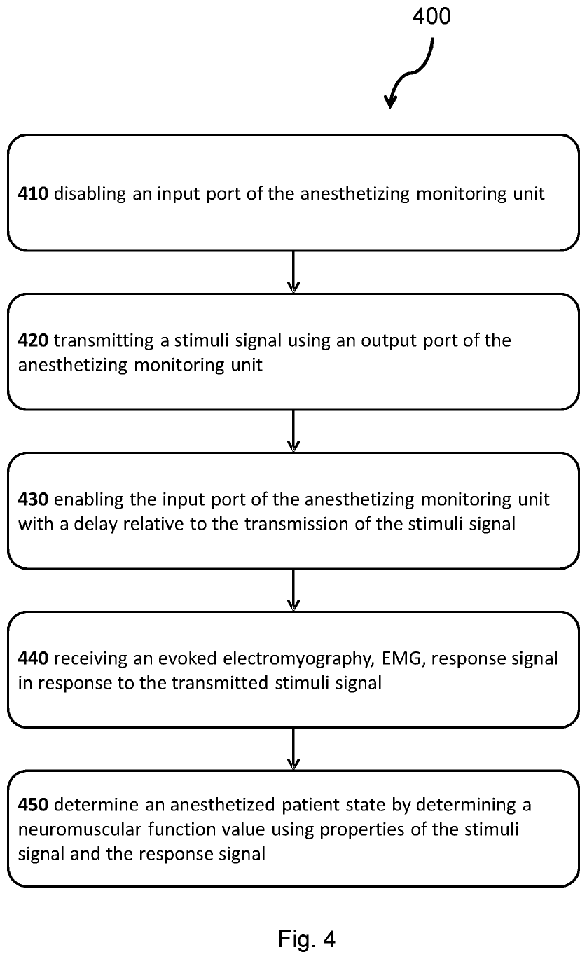

The response signal S_{Response} has ideally constant peak voltage amplitude of A_{Response}, as most of the non-linear artifact has been removed by the disclosed method. As described further in relation to FIG. 1, the response signal S_{Response} is obtained from the anesthetized patient 130 by receiving electrodes 121 attached to the anesthetized patient 130. The response signal S_{Response} is received using the input port 111 of the anesthetizing monitoring unit 110. The neuromuscular blocked patient state may then be determined by determining a neuromuscular function value using properties of the stimuli signal such as amplitude A_{Stimuli}, and the response signal S_{Response}, such as amplitude A_{Response}, as further described in relation to FIG. 4.

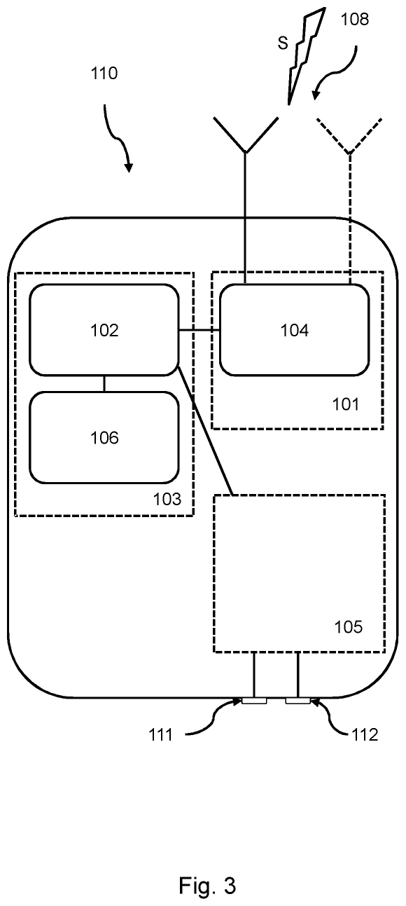

FIG. 3 illustrates an anesthetizing monitoring unit 110 according to one or more embodiments of the present invention. The anesthetizing monitoring unit 110 comprises processing circuitry 103. The processing circuitry 103 may comprise a processor 102 communicatively coupled to a memory 106, said memory 106 comprising instructions executable by said processor, whereby said anesthetizing monitoring unit 110 is operative to perform the method of any of the embodiments described herein.

The processing circuitry 103 may in one optional embodiment be communicatively coupled to a communication interface 101, e.g. comprising one or more transceivers 104. The communication interface 101 may be operative to receive information, such as a data packet, from the processor 102 and generate a wireless signal S for a wireless communication network or to receive the wireless signal S for a wireless communication network 231-233. The communication interface 101 may further be operative to demodulate and/or decode the wireless signal S to a data packet and send to the processor 102. Further, the anesthetizing monitoring unit 110 may further comprise one or more optional antennas 108, as shown in FIG. 2. The antenna/s 108 is/are coupled to the transceiver/s 104 and is/are configured to transmit/emit or receive wireless signals S for a wireless communication network, e.g. transmit a data packet included or comprised in the wireless signal S. The processor and/or a processor unit 102 may be, e.g. processing circuitry and/or a central processing unit and/or processor modules and/or multiple processors configured to cooperate with each-other. The memory 106 may comprise of essentially any suitable memory, such as a ROM (Read-Only Memory), a PROM (Programmable Read-Only Memory), an EPROM (Erasable PROM), a Flash memory, an EEPROM (Electrically Erasable PROM), or a hard disk drive. The communication interface 101 may be configured to send or receive data to other nodes such as servers or other anesthetizing monitoring units. E.g. transmitting or receiving anesthetized patient state/s, neuromuscular function value/s or properties of the stimuli signal S_{Stimuli}, and/or the response signal S_{Response}.

The processing circuitry 103 may in one embodiment be communicatively coupled to a measurement interface 105. The measurement interface 105 is further coupled to the input port 111 and the output port 112. The measurement interface 105 is configured to transmit the stimuli signal S_{Stimuli} in response to a control signal received from the processing circuitry 103. The control signal may e.g. comprise properties of the stimuli signal S_{Stimuli}. The measurement interface 105 is further configured to receive the response signal S_{Response}, detecting properties of the response signal S_{Response} and send the properties of the response signal S_{Response} to the processing circuitry 103. The properties of the response signal S_{Response} may include one or more amplitude values A_{Response}, e.g. voltage amplitude. The measurement interface 105 may further be configured to enable and/or disable the input port 111 in response to a control signal received from the processing circuitry 103.

In one or more embodiments, the anesthetizing monitoring unit 110 may further comprise an input device (not shown in the figure), configured to receive input or indications from a user and send a user-input signal indicative of the user input or indications to the processor and/or a processor unit 102.

In one or more embodiments the anesthetizing monitoring unit 110 may further comprise a display (not shown in the figure) configured to receive a display signal indicative of rendered objects, such as text or graphical user input objects, from the processing circuitry 103 and to display the received signal as objects, such as text or graphical user input objects.

In one embodiment, the display is integrated with the user input device and is configured to receive a display signal indicative of rendered objects, such as text or graphical user input objects, from the processing circuitry 103 and to display the received signal as objects, such as text or graphical user input objects, and/or configured to receive input or indications from a user and send a user-input signal indicative of the user input or indications to the processing circuitry 103.

In Embodiments, the Processing Circuitry 103 is Further Communicatively Coupled to the Input Device and/or the Display.

FIG. 4 shows a block diagram of a method 400 according to one or more embodiments of the present invention. The method 400 is performed by an anesthetizing monitoring unit 110 configured to determine an anesthetized patient state.

In a first example, the anesthetized patient state based on neuro-muscular block may be determined during surgery as any of “insufficient anesthetic level”, “low anesthetic level” or “sufficient anesthetic level”. In a second example, the anesthetized patient state may be determined for the anesthetized patient at recovery after surgery as “normal breathing function”, “capable of sustaining breathing” or “in need of ventilator”.

The determination of the anesthetized patient state may e.g. be based on comparing the response signal S_{Response} to threshold values, as further described below.

The method 400 comprises:

STEP 410: disabling an input port 111 of the anesthetizing monitoring unit 110. As mentioned in relation to FIG. 2, the input port 111 may be enabled and/or disabled in response to a control signal received from the processing circuitry 103. The input port 111 may be enabled and/or disabled by activating a switching unit 1052.

In one example, in order to prevent any electrical artifact from affecting the anesthetizing monitoring unit 110, the input ports 111, 112 are shorted together. In one embodiment including an alternating current, AC, coupled system the input ports 111, 112 may be shorted on the anesthetizing monitoring unit 110 side of the coupling to a bias circuit, providing a low impedance path for fast recovery. In one embodiment including a direct current, DC, coupled monitoring circuit the inputs may be shorted to a common mode reference point or right leg drive potential. The short may be applied by an electro-mechanical switch such as a relay or a semiconductor switch such as a transistor or any other component suitable for disconnecting the input port 111 from the anesthetizing monitoring unit 110 or measurement interface 105 or to connect circuit the input port 111 to any of a ground potential, a common mode reference point or right leg drive potential.

Such an arrangement ensures minimal effect on the response signal when the short is removed, such that any stimulus artifact is hidden from an amplifier comprised in the anesthetizing monitoring unit 110 and the response signal therefore is accurately amplified.

STEP 420: transmitting a stimuli signal S_{Stimuli} using an output port 112 of the anesthetizing monitoring unit 110. The stimuli signal S_{Stimuli}, may be in the form of a pulse wave or pulse train pulse or a plurality of pulse wave or pulse train pulses, wherein each pulse may optionally have constant amplitude A_{Stimuli}. As described further in relation to FIG. 1, the stimuli signal S_{Stimuli}, is delivered to the anesthetized patient 130 by stimulating electrodes 122 attached to the anesthetized patient 130.

STEP 430: enabling the input port 111 of the anesthetizing monitoring unit 110 with a delay AT relative to the transmission of the stimuli signal S_{Stimuli}. The input port 111 may be enabled and/or disabled by activating a switching unit, such as an electronic relay, transistor, thyristor, integrated circuit or other component suitable for disconnecting the input port 111 from the anesthetizing monitoring unit 110 or measurement interface 105 or to short circuit the input port 111 to ground potential. The input port 111 may be enabled at time T_{enable} with a delay AT relative to an end time T_{stimuli_end} of a pulse comprise in the stimuli signal S_{Stimuli}, as further described in relation to FIG. 2A and FIG. 2B.

The delay AT may in embodiments be in the range of 1-10 milliseconds, more preferably in the range of 3-4 milliseconds and most preferably in the range of 1-2 milliseconds. The delay AT may be selected based on a determined nerve conduction of the patient, e.g. slow, normal or fast.

STEP 440: receiving an evoked electromyography, EMG, response signal S_{Response} in response to the transmitted stimuli signal S_{Stimuli}. As described further in relation to FIG. 1, the response signal S_{Response} is obtained from the anesthetized patient 130 by receiving electrodes 121 attached to the anesthetized patient 130 and received by the input port 111.

STEP 450: determine an anesthetized patient state by determining a neuromuscular function value using properties of the stimuli signal S_{Stimuli}, and the response signal S_{Response}. As mentioned above, the anesthetized patient state may be determined as a selection of any of the statuses “normal breathing function”, “capable of sustaining breathing”, “in need of ventilator”, “insufficient anesthetic level”, “low anesthetic level” or “sufficient anesthetic level” but not limited thereto.

Additionally or alternatively, the anesthetized patient state may be determined as a selection of any of the statuses “X % of a reference neuromuscular transmission level” or “N responses out of M transmitted stimuli received” but not limited thereto.

The neuromuscular function value may be determined by stimulating an accessible peripheral motor nerve of the anesthetized patient with the stimuli signal S_{Stimuli}, via stimulating electrodes 122. The evoked response, e.g. the evoked response of the skeletal muscle or muscles innervated by the stimulated motor nerve, may then be recorded by the evoked electromyography, EMG, response signal S_{Response}. The anesthetized patient state may then be determined by comparing properties the response signal S_{Response} to threshold values, e.g. predetermined and stored in memory or properties historical response signals received prior to the current or latest response signal S_{Response}. Alternatively or additionally, the anesthetized patient state may then be determined as a ratio of the values A_{Stimuli}, A_{Response} or a response signal pulse count, where A_{Stimuli}, A_{Response} represent amplitude values of a respective signal.

In one embodiment, the properties of the stimuli signal S_{Stimuli}, and the response signal S_{Response} include amplitude values A_{Stimuli}, A_{Response}, such as voltage, power or current amplitude values. The neuromuscular function value may then be determined as a quota of amplitude value/s of the stimuli signal S_{Stimuli}, and amplitude value/s of the response signal S_{Response}. The anesthetized patient state may then be determined by comparing the quota of amplitude value/s to a set of thresholds. The thresholds may be predetermined and stored in memory or derived from historical response signals received prior to the current or latest response signal S_{Response}, e.g. comparing amplitude value/s to individual historical amplitude value/s or aggregated amplitude value/s, e.g. averaged historical amplitude value/s from previously received response signals.

In one example, the set of thresholds include, for the ratio 100*(A_{Response}/A_{Stimuli}), 0-39% indicating an anesthetized patient state of “in need of ventilator”, 40-89% indicating an anesthetized patient state of “capable of sustaining breathing” and 90% indicating an anesthetized patient state of “normal breathing function”.

In one example, the set of thresholds include, for the ratio A_{Response}/A_{Stimuli}, 0-0.39 indicating an anesthetized patient state of “in need of ventilator”, 0.40-0.89 indicating an anesthetized patient state of “capable of sustaining breathing” and 0.90 indicating an anesthetized patient state of normal breathing function”.

In one example, the set of thresholds include, for the ratio 100*(A_{Response}/S_{Stimuli}) 0-39% indicating an anesthetized patient state of “sufficient anesthetic level”, 40-89% indicating an anesthetized patient state of “low anesthetic level” and 90% indicating an anesthetized patient state of “insufficient anesthetic level”.

In one example, the set of thresholds include, for the ratio A_{Response}/A_{Stimuli}, 0-0.39 indicating an anesthetized patient state of “insufficient anesthetic level”, 0.40-0.89 indicating an anesthetized patient state of “low anesthetic level” and 0.90 indicating an anesthetized patient state of “insufficient anesthetic level”.

In one example, the neuromuscular function value is determined as a quota of amplitude values (A_{Response}/A_{Stimuli})=0.9 and the anesthetized patient state is determined as 90% of a reference neuromuscular transmission level, the reference level being 100% or full neuromuscular transmission level.

In one example, the neuromuscular function value is determined as a count of two (2) received response pulses out of four (4) transmitted stimuli pulses and the anesthetized patient state is determined as 2 responses out of 4 transmitted stimuli received.

In one example, determining a neuromuscular function value includes stimulating a motor nerve with a plurality of temporally distinct stimuli, e.g. pulses comprised in the stimuli signal S_{Stimuli}. After each stimulus of the motor nerve, the muscle response in the muscle(s) innervated by the stimulated motor nerve is recorded as an evoked response comprised in the response signal S_{Response} The recorded evoked muscle responses following the application of the plurality of stimuli are evaluated to provide an anesthetized patient state. Each stimulus of the plurality is sufficient to cause an evoked muscle response under normal physiological conditions. As muscle relaxants are administered to patients, the evoked muscle response decreases. Determining the neuromuscular function value may include determining a ratio of amplitude of a particular recorded muscle response to the amplitude of a muscle response resulting from any subsequent or previous response pulse to characterize the neuromuscular function value, which is related to the degree of muscle function or blockade. In some implementations, evaluation of the muscle responses may include determining a ratio of the amplitude of a muscle response from a subsequent pulse to the amplitude of the muscle response from a previous pulse. A neuromuscular function value less than 1.0 indicates the presence of neuromuscular blockade or reduced neuromuscular function in the anesthetized patient.

In one example, one or more of the subsequent pulses do not produce an evoked muscle response. When the subsequent and/or first pulse does not produce an evoked muscle response, the determined ratio is zero indicating presence of neuromuscular blockade in the subject. Optionally, as a supplemental measure, the number of subsequent pulse evoking a muscle response may be counted and determined as the neuromuscular function value.

In one example, the neuromuscular function value is determined as a ratio of the amplitude of the muscle response related to the fourth pulse to the amplitude of the muscle response related to the first pulse of a plurality of stimuli or pulses comprised in the stimuli signal S_{Stimuli}. Although it is not required, in some implementations, the fourth pulse may be the fourth sequential pulse and the first pulse may be the first pulse in the plurality of sequential pulses. Optionally, the ratio is determined as a ratio of the amplitude of the muscle response related to the fifth or greater pulse to the amplitude of the muscle response related to the first pulse. For example, the ratio is optionally determined from the amplitude of the muscle response related to the sixth, seventh, eighth, ninth, or tenth pulse to the amplitude of the muscle response related to the first pulse. Regardless of which number subsequent pulse is used, the ratio is zero if there is no muscle response related to the first and/or the subsequent pulse of the plurality of pulses.

The method optionally further includes identifying one or more stimuli of the plurality of temporally distinct stimuli that caused an evoked muscle response and enumerating them to determine a count. The count can be determined subsequent to determining a zero value ratio. Optionally, the count is zero. A count of zero indicates that none of the one or more of the plurality of stimuli used to determine the count caused an evoked muscle response.

If the ratio or the count is zero, the method optionally comprises stimulating the motor nerve in a tetanic protocol. A tetanic protocol may optionally comprise delivering a plurality of stimuli at a rate that is high enough to cause fusion of the individual evoked muscle responses into a single sustained muscle contraction. Optionally, this may be a rate greater than 30 stimuli per second. A neuromuscular function value based on the ratio of the amplitude of the last evoked response to the amplitude of the first evoked response may be calculated, and a neuromuscular function value greater than 0.9 demonstrates that the anesthetized patient state can be determined to “normal breathing function”. Alternatively, because there may be some amplitude variation in the evoked muscle responses at the beginning of the tetanic stimulation, a ratio of the amplitude of any response toward the end of the stimulation to the amplitude of any response toward the beginning of the stimulation may be calculated.

In one embodiment, the motor nerve is optionally stimulated with a plurality of temporally spaced supplemental stimuli or pulses. After each stimulation of the motor nerve, the muscle responses of the muscle innervated by the stimulated motor nerve are recorded. The number of evoked muscle responses produced by the temporally spaced subsequent stimuli is used to determine a post-tetanic count and indicates an “X % of reference neuromuscular transmission”, where X % indicates a percentage of the number of evoked muscle responses to the temporally spaced supplemental stimuli or pulses.

In one embodiment, the neuromuscular function value is determined by stimulating a motor nerve to cause an evoked muscle response. The evoked muscle response is recorded. A peak of the recorded evoked muscle response is identified. The amplitude of the peak from a baseline is determined. The measured amplitude from the baseline is compared to a control amplitude, determined from prior stimuli, to indicate a change in the neuromuscular function value is determined or that the desired the neuromuscular function value has been maintained.

In one embodiment, the method 400 further comprises displaying the anesthetized patient state to a user of the anesthetizing monitoring unit 110.

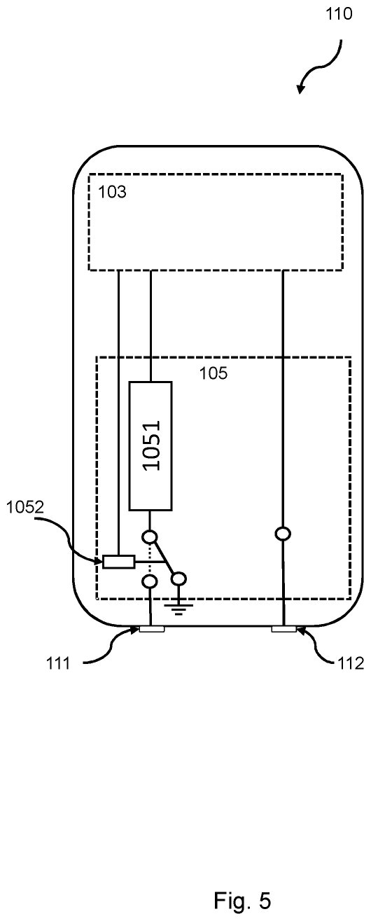

FIG. 5 illustrates an anesthetizing monitoring unit 110 according to one or more embodiments of the present invention. The measurement interface 105 may further comprise a analog to digital converter, A/D, 1051 configured to receive the response signal S_{Response}, sample the response signal S_{Response} to obtain properties of the response signal S_{Response}, such as a amplitude value indicative of the response signals amplitude A_{Response}. The measurement interface 105 may further comprise a switching unit 1052 configured to enable and/or disable input port 111 in response to a control signal received from the processing circuitry 103. The input port 111 may be enabled and/or disabled by activating/deactivating the switching unit 1052. The switching unit 1052 may be e.g. an electronic relay, transistor, thyristor, integrated circuit or other component suitable for disconnecting the input port 111 from the anesthetizing monitoring unit 110 or measurement interface 105 or to short circuit the input port 111 to ground potential.

In one embodiment, a computer program is provided comprising computer-executable instructions for causing an anesthetizing monitoring unit (110), when the computer-executable instructions are executed on a processing unit comprised in the anesthetizing monitoring unit (110) to perform the method 400 described herein.

In one embodiment, a computer program product comprising a computer-readable storage medium, the computer-readable storage medium having the computer program above embodied therein. The memory and/or computer-readable storage medium referred to herein may comprise of essentially any memory, such as a ROM (Read-Only Memory), a PROM (Programmable Read-Only Memory), an EPROM (Erasable PROM), a Flash memory, an EEPROM (Electrically Erasable PROM), or a hard disk drive.

Moreover, it is realized by the skilled person that the anesthetizing monitoring unit 110 may comprise the necessary communication capabilities in the form of e.g., functions, means, units, elements, etc., for performing the present solution. Examples of other such means, units, elements and functions are: processors, memory, buffers, control logic, mapping units, multipliers, decision units, selecting units, switches, inputs, outputs, antennas, amplifiers, receiver units, transmitter units, DSPs, MSDs, encoder, decoder, power supply units, power feeders, communication interfaces, communication protocols, etc. which are suitably arranged together for performing the present solution.

Especially, the processor/processing means of the present disclosure may comprise one or more instances of processing circuitry, processor modules and multiple processors configured to cooperate with each-other, Central Processing Unit (CPU), a processing unit, a processing circuit, a processor, an Application Specific Integrated Circuit (ASIC), a microprocessor, a Field-Programmable Gate Array (FPGA) or other processing logic that may interpret and execute instructions. The expression “processor” and/or “processing means” may thus represent a processing circuitry comprising a plurality of processing circuits, such as, e.g., any, some or all of the ones mentioned above. The processor/processing means may further perform data processing functions for inputting, outputting, and processing of data comprising data buffering and device control functions, such as call processing control, user interface control, or the like.

Finally, it should be understood that the invention is not limited to the embodiments described above, but also relates to and incorporates all embodiments within the scope of the appended independent claims.

1. A method performed by an anesthetizing monitoring unit configured to determine an anesthetized patient state, the method comprising:
disabling an input port of the anesthetizing monitoring unit,
transmitting a stimuli signal (S_{Stimuli}) using an output port of the anesthetizing monitoring unit,
enabling the input port of the anesthetizing monitoring unit with a delay (ΔT) relative to the transmission of the stimuli signal (S_{Stimuli}),
receiving an evoked electromyography, EMG, response signal (S_{Response}) in response to the transmitted stimuli signal (S_{Stimuli}),
determine an anesthetized patient state by determining a neuromuscular function value using properties of the stimuli signal (S_{Stimuli}) and the response signal (S_{Response}).

2. The method according to claim 1, wherein the delay (ΔT) is in the range of 1-10 milliseconds, more preferably in the range of 3-4 milliseconds and most preferably in the range of 1-2 milliseconds.

3. The method according to claim 1, wherein the anesthetized patient state is a selection of any of “normal breathing function”, “capable of sustaining breathing”, “in need of ventilator”, “insufficient anesthetic level”, “low anesthetic level” or “sufficient anesthetic level”.

4. The method according to claim 3, wherein the properties of the stimuli signal (S_{Stimuli}) and the response signal (S_{Response}) include amplitude values (A_{Stimuli}, A_{Response}) and wherein the neuromuscular function value is determined as a quota of amplitude value/s of the stimuli signal (S_{Stimuli}) and amplitude value/s of the response signal (S_{Response}).

5. The method according to claim 4, wherein the anesthetized patient state is determined by comparing the quota of amplitude value/s to a set of predetermined thresholds.

6. The method according to claim 1, further comprising displaying the anesthetized patient state to a user of the anesthetizing monitoring unit.

7. An anesthetizing monitoring unit configured to determine an anesthetized patient state, the anesthetizing monitoring unit comprising:
an input port,
an output port, and
processing circuitry being configured to perform the method according to claim 1.

8. An anesthetizing monitoring system configured to determine an anesthetized patient state, the anesthetizing monitoring system comprising:
an anesthetizing monitoring unit comprising an input port and an output port,
stimulating electrodes electrically coupled to the output port and being configured to receive a stimuli signal (S_{Stimuli}) from the output port and deliver the stimuli signal (S_{Stimuli}) to the anesthetized patient,
receiving electrodes electrically coupled to the input port and being configured to obtain an evoked electromyography, EMG, response signal (S_{Response}), in response to the stimuli signal (S_{Stimuli}), from the anesthetized patient, the anesthetizing monitoring unit being configured to perform the method according to claim 1.

9. A computer program is provided comprising computer-executable instructions for causing an anesthetizing monitoring unit, when the computer-executable instructions are executed on a processing unit comprised in the anesthetizing monitoring unit, to perform the method according claim 1.

10. A computer program product comprising a computer-readable storage medium, the computer-readable storage medium having the computer program according to claim 9 embodied therein.

11. A carrier containing the computer program according to claim 9, wherein the carrier is one of an electronic signal, optical signal, radio signal, or computer readable storage medium.
